SEQUENCE LISTING (20 sequences)

SEQ ID NO: 1 - DNA, 30 bp, Artificial Sequence
  Primer B4600
gataactagtcctggtccaccggggtcaag

SEQ ID NO: 2 - DNA, 36 bp, Artificial Sequence
  Primer B4601
gctcagatcttccttaagtcagtcgcgcaggttcag

SEQ ID NO: 3 - DNA, 37 bp, Artificial Sequence
  Primer B4602
aggaagatctgagctagctcggaccagaacgaaaaag

SEQ ID NO: 4 - DNA, 29 bp, Artificial Sequence
  Primer B4603
gatactcgaggcggatgaacattgaggtg

SEQ ID NO: 5 - DNA, 72 bp, Artificial Sequence
  Primer B4604
gatacttaagaaaacaaactaaagcgcccttgtggcgctttagttttatactactgagaaaaatctggattc

SEQ ID NO: 6 - DNA, 43 bp, Artificial Sequence
  Primer B4605
gattttcatcaatactcctggatcccgttaattcgaagagtcg

SEQ ID NO: 7 - DNA, 43 bp, Artificial Sequence
  Primer B4606
cgactcttcgaattaacgggatccaggagtattgatgaaaatc

SEQ ID NO: 8 - DNA, 29 bp, Artificial Sequence
  Primer B4607
gataagatcttcaggagccttgattgatc

SEQ ID NO: 9 - DNA, 29 bp, Artificial Sequence
  Primer B4608
gataagatctgcgcaacgcaattaatgtg

SEQ ID NO: 10 - DNA, 64 bp, Artificial Sequence
  Primer B4609
gatagctagcagtcaaaagcctccggtcggaggcttttgactttatttttgacaccagaccaac

SEQ ID NO: 11 - DNA, 28 bp, Artificial Sequence
  Primer B4665
gatagctagcttggccagaaagaaggcg

SEQ ID NO: 12 - DNA, 37 bp, Artificial Sequence
  Primer B4666
gatacatatgtcggtacctattcgcccaaaagaaaag

SEQ ID NO: 13 - DNA, 29 bp, Artificial Sequence
  Primer B4667
gatacatatgtcaatactcctgatttttg

SEQ ID NO: 14 - DNA, 29 bp, Artificial Sequence
  Primer B4668
gatagctagcaatgaaatggacgcggatc

SEQ ID NO: 15 - DNA, 65 bp, Artificial Sequence
  Primer B4669
gataggtaccgatctagtcaaaagcctccgaccggaggcttttgactttagtacttgccgcctag

SEQ ID NO: 16 - DNA, 29 bp, Artificial Sequence
  Primer B4670
gataccatggcaaattatcaaaacgcatc

SEQ ID NO: 17 - DNA, 28 bp, Artificial Sequence
  Primer B4671
gataccatggtagttcctcgataagtcg

SEQ ID NO: 18 - DNA, 39 bp, Artificial Sequence
  Primer B4672
gatacatatgcctagggatctgaccgaccgatctactcc

SEQ ID NO: 19 - DNA, 29 bp, Artificial Sequence
  Primer B4673
gataggtaccttagcgccattcgccattc

SEQ ID NO: 20 - DNA, 29 bp, Artificial Sequence
  Primer B4674
gataggtaccgcgcaacgcaattaatgtg

(19) United States
(12) Patent Application Publication
(10) Pub. No.: US 2017/0304378 A1
(43) Pub. Date: Oct. 26, 2017

(54) MODIFYING BACTERIOPHAGE USING BETA-GALACTOSIDASE AS A SELECTABLE MARKER

(71) Applicant: Phico Therapeutics Ltd, Cambridge (GB)

(72) Inventors: Heather FAIRHEAD, Cambridge (GB); Adam WILKINSON, Royston (GB); Neil ANDERSON, Bishop's Stortford (GB); Katy PITTS, Royston (GB); Anne BARNARD, Cambridge (GB); Emmanuele SEVERI, Cambridge (GB)

(21) Appl. No.: 15/517,718

(22) PCT Filed: Oct. 8, 2015
(86) PCT No.: PCT/EP2015/073297

§ 371 Date: Apr. 7, 2017

(30) Foreign Application Priority Data
Oct. 8, 2014 (GB) 1417811.5

(51) Int. Cl. A61K 35/76; C12N 7/00; A61K 35/00

(52) U.S. Cl. CPC A61K 35/76; C12N 7/00; C12N 2795/00041; A61K 35/00; C12N 2795/00043; C12N 2795/00032

(57) ABSTRACT

A method for modifying the genome of a target phage is described. Compositions comprising such modified phage are also described. The compositions may be formulated as a medicament, which are useful for human treatment and may treat various conditions, including bacterial infections.

The present invention relates to a method for modifying the genome of a target phage.

### BACKGROUND TO THE INVENTION

Bacteriophage are the most abundant organisms in the world with an estimated 10³⁰ present at any one time. Bacteriophage reportedly can inhabit every imaginable environment (Brabban et al., 2005), thus providing a huge reservoir of biological diversity for use in biotechnology. Phage have been used in a variety of applications, such as phage display to characterise protein-protein interactions (Smith and Petrenko, 1997), diagnostic tests for the rapid identification of bacterial pathogens (Dobozi-King et al., 2005), and in the treatment of bacterial infections by “phage therapy” (Harper et al., 2011). Another use of phage is as the basis for modification to make tailored gene delivery vehicles, which can be used for the delivery of genes encoding toxic proteins to target pathogenic bacteria. Such an approach is described in the SASPject system (WO2009/019293), in which bacteriophage are engineered to be non-lytic, thus ultuimately non-viable, and to carry a SASP gene expression cassette, which is delivered into the targeted bacteria, which leads to rapid SASP expression. SASP are Small Acid-soluble Spore Proteins, which protect the DNA of Gram positive bacterial endospores during dormancy. However, upon expression of SASP in vegetative cells, rapid binding of SASP to the cell's DNA in a non-sequence specific manner (Nicholson et al., 1990) leads to rapid cell death.

Phage can be broadly split into temperate and non-temperate phage (Abedon, 2008). Temperate phage are able to exist in two distinct lifestyles. In one lifestyle, temperate phage replicate “lytically”—they infect the host cell, replicate and make new phage progeny, a process which ends in the lysis of the cell and the release of mature phage particles. In the other lifestyle, temperate phage infect the cell and integrate into the host cell genome, usually at specific attachment sites, to become “prophage”. In so doing, they become a transient part of the host cell's genome, and are replicated together with the host cell's DNA. Integrated prophage are generally harmless to their host cell whilst in this integrated state, and can often provide selective advantage to the cell, by providing extra genes to the cell, e.g. CTX toxin genes are provided by CTX prophage to Vibrio cholera, increasing the virulence of such strains compared to non-toxin gene carrying strains (Waldor and Mekalanos, 1996). In contrast, non-temperate phage, otherwise known as “lytic” phage, are only able to replicate in the lytic lifestyle described above they cannot integrate into host cell DNA and therefore never become part of the host cell genome: Henceforth such phages will be described as “obligately lytic” to distinguish them from temperate phage which are capable of both lytic and prophage replication.

When choosing phage for genetic modification, for instance when such phage are to act as delivery vectors for a gene encoding an anti-bacterial protein, such as SASP, the amenability of the phage to genetic modification is an important factor. Broadly speaking, temperate bacteriophage are easily genetically modified, providing that the bacterial host species can be manipulated by standard molecular genetic techniques involving recombination and resistance marker selection, or a recombineering system (Thomason et al., 2014).

Temperate phage, in the form of lysogens which carry integrated phage DNA as a prophage, can be engineered to carry exogenous DNA linked to any of a wide array of selectable markers, such as antibiotic or heavy metal resistance markers. Such markers may be linked to exogenous DNA and flanked by regions of prophage DNA, and cloned into suitable vectors which are not replicative (suicide vectors) in the bacterial host (lysogen). Upon introduction of such plasmids into the bacterial lysogen, by common methods such as conjugation or chemical or electro-transformation, recombinants which have integrated via the homologous sequences present on the plasmid, may be selected via the resistance marker linked to the exogenous DNA. Counter-selectable markers can also be used in engineering temperate phage. Recombinant phage which have retained the resistance marker can be screened by common methods such as PCR. Alternatively a counter-selectable marker, such as sacB, can be engineered into the backbone of the plasmid used for engineering such phage, and by selecting for the resistance marker linked to the exogenous DNA but against the counter-selectable marker, recombinant prophage can be isolated carrying only the exogenous DNA. The genotype can be confirmed by PCR. Such vectors are commonly available. Such engineered phage can be induced from the lysogenised strain, for example by the addition of Mitomycin C (Williamson et al., 2002) at which point the phage excise from the host chromosome and enter their lytic phase such that the retained marker can no longer be selected, but the marker and exogenous DNA remain in the phage genome.

Isolation of genetically manipulated lytic phage, however, cannot be achieved using the same methods described above. For example it is impossible to use conventional positive selection in order to isolate engineered obligately lytic phage, such as antibiotic and heavy metal resistance markers, which confer resistance to bacteria, cannot be selected due to the obligately lytic lifestyle of the phage. The DNA of the phage never becomes part of the host cell genome and therefore selectable resistance markers which convey resistance to the host are not selectable when located on obligately lytic phage.

Some techniques have been developed for the engineering of lytic phage. One such example is the BRED technique (Bacteriophage Recombineering of Electroporated DNA) (Marinelli et al., 2008), which uses a “recombineering” approach, and has been described for the engineering of Mycobacterium phage. Recombineering methods for the manipulation of bacterial genomes were first described in the λ Red system (Yu et al., 2000). In this technique the recombination proteins Exo and Beta catalyse the efficient recombination of linear DNA sequence introduced into host cell via transformation. The BRED approach similarly utilises recombination promoting proteins—the RecE/RecT-like proteins gp60 and gp61 from a Mycobacterium bacteriophage—to promote high levels of recombination when phage genomes are co-transformed with linear “targeting” DNA fragments into M. smegmatis cells. Recombinant phage are then screened and identified with relative ease due to the high efficiency of recombination. However, such an approach relies upon the development of an efficient recombineering system in the chosen bacterial species. Furthermore, phage genomes are large (Hendrix, 2009) and transformation of large DNA molecules is inefficient even in readily transformable bacteria such as E. coli (Sheng et al, 1995), and efficient transformation techniques have not been developed for many bacterial species.

Specific techniques have been developed for the engineering of certain bacteriophage. For instance a technique known as RIPh (Rho*-mediated inhibition of phage replication) has been developed for phage T4 (Pouillot et al., 2010). Early genes essential to phage replication are transcribed as concatenated run-through RNAs requiring the host transcription terminator factor Rho for the production of the early proteins. It was found that engineering E. coli to contain an inducer-controlled overexpressed mutated copy of Rho, called Rho*, inhibits production of the early T4 proteins and thus reversibly inhibits T4 phage replication, but has a minimal effect on host cell viability. In this state the T4 genome does not replicate and there is not continuation of the phage lifecycle through to mature phage synthesis and lysis, but it is not lost from the cell, and is a substrate for recombination. In the RIPh technique, the X Red system is used to target recombination into the T4 genome whilst it is in this stable suspended state. Removal of the inducer allows the phage to continue its lifecycle and mature, engineered, phage are formed.

Another example of specific obligately lytic phage engineering systems is found in T7 phage. The E. coli genes trxA and cmk are required for the propagation of phage T7, but are not required for the growth of the host cell (Qimron et al, 2006; Mark, 1976). Therefore T7 could be engineered to carry either of these “marker” genes, by selecting recombinant phage on engineered host cells that lack the marker genes. However, in both the T4 and T7 example, quite specific and detailed knowledge of the phage's replication machinery, or the host cell genes specifically required for phage replication, is required.

It would be desirable to have a technique for modifying the genome of phage which may be used for both temperate and lytic phage, and which does not rely upon specific detailed knowledge of the genes involved in the replication pathway of the phage or the genes of the host cell required for phage propagation in the cell. It would further be desirable for such a technique to be broadly applicable to phage from any bacterial species.

### SUMMARY OF THE INVENTION

The present invention provides A method for modifying the genome of a target phage, which comprises
(a) providing a vector which contains a phage-targeting region which comprises a phage genome modifying element and encodes β-galactosidase or a subunit thereof;
(b) mixing the vector with the target phage so as to modify the genome of the target phage;
(c) propagating the resultant phage on a reporter host cell in the presence of a β-galactosidase substrate labelled with a reporter label under conditions to release the label in the presence of (β-galactosidase activity; and
(d) harvesting phage exhibiting 13-galactosidase activity in the reporter host cell.

It has surprisingly been found that lacZ (encoding (β-galactosidase) can be used as a selectable marker to isolate recombinant phage and to identify recombinant phage, for example by propagation on a bacterial lawn in the presence of a labelled substrate which is typically a labelled galactose or analogue thereof such as S-Gal or X-Gal. This invention is particularly useful for the genetic modification of obligately lytic phage which cannot form lysogens, as it provides a means of genetic selection that is not reliant upon selecting characteristics of the host cell, and instead selects for a characteristic inherited by the phage in its lytic state.

This technique does not rely upon specific detailed knowledge of the genes involved in the phage's replication pathway and/or the host cell gene(s) required for phage propagation in the cell, therefore not requiring undue experimentation on the phage prior to manipulation. Furthermore, this technique is broadly applicable to phage from any bacterial species. This can be readily performed by those skilled in the art, as directed by this application.

The mixing of the vector with the target phage may take place in a host cell which has been infected by the target phage. The vector, which may be a plasmid, is introduced into the host cell, which host cell is a host for the target phage. The target phage then infects the cell and replication follows. Modification of the genome of the target phage may then result by recombination.

The phage targeting region of the vector may be designed to promote recombination. Preferably, the target phage genome includes a first target sequence and a second target sequence. The phage-targeting region of the vector is flanked by first and second flanking sequences homologous to the first and second target sequences of the target phage genome to allow the recombination to take place. In this way, the genome of the target phage is modified.

The genome of the target phage may be modified by incorporation of an exogenous DNA sequence therein, by incorporation of a mutation such as a point mutation, or by creating a deletion. Combinations of these modifications may also be made.

Because the first and second flanking sequences of the phage targeting region are homologous to the first and second target sequences of the target phage genome, once the host cell for the target phage contains both the vector and the target phage, the phage can replicate and recombination can take place at the pairs of sequences homologous with one another. Following recombination, only those resultant phage carrying sequence encoding β-galactasidase or a subunit thereof will release label in the reporter host cell. This enables selection of desired resultant phage containing the modified genome.

The first and second target sequences of the target phage genome may be contiguous or non-contiguous. In one arrangement, the first and second target sequences of the target phage general are non-contiguous. According to this arrangement, where a recombination event occurs between the first and second flanking sequences and the first and second target sequences the region of DNA between the first and second target sequences is excised from the target phage genome resulting in a deletion. Advantageously, where the first and second target sequences of the target phage genome flank a phage gene or part thereof, such deletion results in inactivation of the gene following recombination. In one arrangement the phage gene is a lysis gene such as an endolysin. In this way lytic target phage can be rendered non-lytic.

Where modification of the genome of the target phage involves incorporation of an exogenous DNA sequence, the phage-targeting region of the vector further comprises an exogenous DNA sequence for incorporation into the genome of the target phage. Because the exogenous DNA sequence and the sequence encoding β-galactosidase or subunit thereof both fall within the first and second flanking sequences of the phage-targeting region, recombination of the phage to select for β-galactosidase will result in incorporation of the exogenous DNA sequence in the resultant phage. According to this arrangement, the first and second target sequences of the target phage genome may be contiguous or non-contiguous. Where they are non-contiguous, incorporation of the exogenous DNA sequence will simultaneously result in deletion of a region of the genome. Where the first and second target sequences are positioned in a phage gene or where they flank a phage gene or part thereof, incorporation of the exogenous DNA sequence will simultaneously result in inactivation of the gene following recombination.

Where a mutation, such as a point mutation is needed to be incorporated into the target phage, at least one of the first and second flanking sequences in the phage-targeting region would contain the mutation as compared with the first and second target sequences of the target phage genome. Upon replication and recombination of the phage the genome of the target phage would be modified so as to incorporate the mutation. In this way, mutant phage would be selected on the reporter host cell because the mutant phage would contain the sequence encoding (β-galactosidase or a subunit thereof and could then be screened for the presence of the mutation.

As described above, introduction of such mutations could be combined with the incorporation of exogenous DNA sequence optionally together with a deletion in the target phage.

The phage-targeting region of the vector encodes β-galactosidase or a subunit thereof β-galactosidase contains alpha and omega subunits, each of which is inactive without the other. β-galactosidase is encoded by the lacZ gene and the alpha and omega subunits are encoded by lacZ alpha and lacZΔM15 respectively. In accordance with the method of the invention, use of the complete lacZ gene as a marker enables selection of the resultant phage on a reporter host cell. Where one or other of the inactive subunits is used as a marker for the resultant phage, the other of the subunits must be encoded by the host cell so that β-galactosidase activity is detected in the presence of the recombinant phage. Advantageously, the phage targeting region encodes the alpha subunit of β-galactosidase because this is relatively small (180 base pairs). In this arrangement the host cell contains the lacZΔM15 encoding the omega subunit.

The reporter label may be any detectable label and is typically an organic moiety covalently linked to galactose or an analogue thereof. Suitable labels include colourimetric labels.

Surprisingly it has been found that lacZ (encoding β-galactosidase, henceforth “LacZ”) can be used as a marker to isolate recombinant phage in combination with the colourimetric substrate S-Gal (Sigma, S9811). The addition of the lacZ gene to the genome of a phage can be used as a marker to identify recombinant phage by propagating on a bacterial lawn in the presence of S-Gal, with recombinant plaques, carrying lacZ and other genetic changes, being easily identifiable compared to non recombinant plaques (FIG. 4). Other commonly used P-galactosidate substrates, such as X-Gal, yield faint blue plaques. It has not previously been thought possible to use lacZ as a marker for high throughput screening for recombinant phage. This invention is particularly useful for the genetic modification of obligately lytic phage which cannot form lysogens, as it provides a means of genetic selection that is not reliant upon selecting characteristics of the host cell, and instead selects for a characteristic inherited by the phage in its lytic state.

A preferred embodiment of this invention is to use the full length (3075 bp) E. coli lacZ gene as a selectable marker. A particularly preferred embodiment of this invention is to use the lacZα sequence as a selectable marker. It is preferable to incorporate the truncated lacZα sequence (180 bp encoding the LacZ α peptide) into the phage genome, selecting on a bacterial host with the lacZΔM15 allele (encoding the lacZ w-peptide) at a suitable location in its genome, to facilitate cloning. The α- and ω-LacZ peptides are inactive as individual proteins, but when expressed in the same cell a functional β-galactosidase enzyme is formed (Welpley et al, 1981). Thus, using lacZα instead of lacZ minimises the size of the inserted marker DNA into the bacterial genome.

In each case a promoter is selected for expression of the lacZ/lacZα cassette. Preferred promoters are active in the host cell. A particularly preferred promoter is the lac promoter.

This approach can be used as a technique for the manipulation of a lytic phage genome if the lacZ or lacZα sequence is introduced alongside adjustments to the phage genome.

In one arrangement of this invention, lacZ/lacZα selection can be used to identify phages carrying exogenous DNA sequence(s): sequences of homology flanking the site of insertion in the phage can be cloned into a plasmid capable of replicating in the chosen bacterial host; lacZ/lacZα can be cloned between the homology arms together with exogenous DNA sequence(s). The plasmid would be transformed into the bacterial host for the phage, and bacteria carrying the engineered plasmid would be infected by phage, and a phage lysate isolated. The phage lysate would be used to identify lacZ/lacZα expressing plaques as black plaques on a suitable strain (carrying the lacZαM15 allele encoding the w-peptide in the case of lacZα recombinants) in the presence of S-Gal. Black plaques can be picked and tested by PCR for the presence of the expected recombinant sequence.

In another arrangement of this invention, the lacZ/lacZα marker can be removed, to leave markerless insertions. This can be achieved by making versions of the plasmids described above which are isogenic other than lacking the lacZ/lacZα cassette: sequences of homology flanking the site of insertion in the phage can be cloned into a plasmid capable of replicating in the chosen bacterial host; the DNA sequence(s) is cloned between the homology sequences. The plasmid would be transformed into the bacterial host for the phage, and bacteria carrying the engineered plasmid would be infected by phage, and a phage lysate isolated. The phage lysate would be used to identify plaques which have lost lacZ/lacZα by plaquing on the lacZΔM15 allele-expressing strain in the presence of S-Gal. Clear plaques can be picked and tested by PCR for the presence of the expected recombinant sequence, and the absence of lacZ/lacZα.

In another arrangement of this invention lacZ/lacZα selection can be used to identify phages with sequences which have been deleted: Non-contiguous sequences of homology flanking the proposed deletion site in the phage can be cloned into a plasmid capable of replicating in the chosen bacterial host; lacZ/lacZα can be cloned between the homology arms. The plasmid would be transformed into the bacterial host for the phage, and bacteria carrying the engineered plasmid would be infected by phage, and a phage lysate isolated. The phage lysate would be used to identify lacZ/lacZα expressing plaques as black plaques on a suitable strain (carrying the lacZΔM15 allele encoding the w-peptide in the case of lacZα recombinants) in the presence of S-Gal. Black plaques can be picked and tested by PCR for the presence of the expected deletion in the phage DNA sequence. The lacZ/lacZα cassette could be removed using derivative plasmids lacking the lacZ/lacZα cassette and subsequently screening for clear plaques as described above.

In another arrangement of this invention lacZ/lacZα selection can be used to identify phages with sequences which carrying point mutations: sequences of homology carrying one or more point mutations in the DNA sequence compared to the targeted phage, and flanking a suitable marker insertion site in the phage, can be cloned into a plasmid capable of replicating in the chosen bacterial host; lacZ/lacZα can be cloned between the homology arms. The plasmid would be transformed into the bacterial host for the phage, and bacteria carrying the engineered plasmid would be infected by phage, and a phage lysate isolated. The phage lysate would be used to identify lacZ/lacZα expressing plaques as black plaques on a suitable strain (carrying the lacZΔM15 allele encoding the w-peptide in the case of lacZα recombinants) in the presence of S-Gal. Black plaques can be picked and tested by PCR and sequencing, to check for the insertion of the desired point mutations. The lacZ/lacZα cassette could be removed using derivative plasmids lacking the lacZ/lacZα cassette and subsequently screening for clear plaques as described above.

In another arrangement, an exogenous DNA sequence can be added to a phage, together with a deletion in the targeted phage, by a combination of the approaches described above. The lacZ/lacZα cassette could be removed by the approach described above.

In another arrangement, an exogenous DNA sequence can be added to a phage, together with a point mutation in the targeted phage, by a combination of the approaches described above. The lacZ/lacZα cassette could be removed by the approach described above.

In another arrangement, an exogenous DNA sequence can be added to a phage, together with a deletion in the targeted phage and a point mutation(s) introduced by a combination of the approaches described above. The lacZ/lacZα cassette could be removed by the approach described above.

In another arrangement, a deletion could be made in the targeted phage and a point mutation(s) introduced by a combination of the approaches described above. The lacZ/lacZα cassette could be removed by the approach described above.

In one embodiment, this invention may be used to identify modified obligately lytic bacteriophage. In another embodiment, the invention may be used to identify modified temperate phage, either as lysogens or during lytic growth. The lacZ/lacZα cassette could be removed by the approach described above.

In one arrangement, this invention may be used to modify obligately lytic bacteriophage. In another arrangement, the invention may be used to modify a temperate phage, during lytic growth.

In a further arrangement, this invention is particularly useful in the engineering of obligately lytic bacteriophage for use as gene delivery vehicles. In a preferred arrangement, this invention could be used to modify lytic phage to carry a gene for an antibacterial protein.

As an alternative to conventional antibiotics, one family of proteins which demonstrate broad spectrum antibacterial activity inside bacteria comprises the α/β-type small acid-soluble spore proteins (known henceforth as SASP). Inside bacteria, SASP bind to the bacterial DNA: visualisation of this process, using cryoelectron microscopy, has shown that SspC, the most studied SASP, coats the DNA and forms protruding domains and modifies the DNA structure (Francesconi et al., 1988; Frenkiel-Krispin et al., 2004) from B-like (pitch 3.4 nm) towards A-like (3.18 nm; A-like DNA has a pitch of 2.8 nm). The protruding SspC motifs interact with adjacent DNA-SspC filaments packing the filaments into a tight assembly of nucleo-protein helices. In 2008, Lee et al. reported the crystal structure at 2.1 A resolution of an α/β-type SASP bound to a 10-bp DNA duplex. In the complex, the α/β-type SASP adopt a helix-turn-helix motif, interact with DNA through minor groove contacts, bind to approximately 6 bp of DNA as a dimer and the DNA is in an A-B type conformation. In this way DNA replication is halted and, where bound, SASP prevent DNA transcription. SASP bind to DNA in a non-sequence specific manner (Nicholson et al., 1990) so that mutations in the bacterial DNA do not affect the binding of SASP. Sequences of a/13-type SASP may be found in appendix 1 of WO02/40678, including SASP-C from Bacillus megaterium which is the preferred α/β-type SASP. WO02/40678 describes the use as an antimicrobial agent of bacteriophage modified to incorporate a SASP gene.

Bacteriophage vectors modified to contain a SASP gene have generally been named SASPject vectors. Once the SASP gene has been delivered to a target bacterium, SASP is produced inside those bacteria where it binds to bacterial DNA and changes the conformation of the DNA from B-like towards A-like. Production of sufficient SASP inside target bacterial cells causes a drop in viability of affected cells.

In particularly preferred embodiments, the method of the present invention may be used to engineer a SASP expression cassette into a phage to create a SASPject vector; this technique could also be used to engineer a SASP expression cassette into a phage and simultaneously delete a lytic gene to create a SASPject vector.

Accordingly, in one arrangement according to the invention, the exogenous DNA comprises a gene encoding an α/β small acid-soluble spore protein (SASP). In this way, the method of the invention may be used to produce a modified bacteriophage capable of infecting target bacteria. The modified bacteriophage includes a SASP which is toxic to the target bacteria, wherein the bacteriophage is typically non-lytic.

The SASP gene may be chosen from any one of the genes encoding the SASP disclosed in Appendix 1 of WO02/40678. In a preferred arrangement the SASP is SASP-C. The SASP-C may be from Bacillus megaterium.

In one aspect, the term ‘SASP’ as used in the present specification refers to a protein with α/β-type SASP activity, that is, the ability to bind to DNA and modify its structure from its B-like form towards its A-like form, and not only covers the proteins listed in appendix 1 of WO02/40678, but also any homologues thereof, as well as any other protein also having α/β-type SASP activity. In an alternative aspect, the term ‘SASP’ as used in the specification refers to any protein listed in appendix 1 of WO02/40678, or any homologue having at least 70%, 75%, 80%, 85%, 90%, 92%, 94%, 95%, 96%, 98% or 99% sequence identity with any one of the proteins listed in appendix 1 of WO02/40678. In another alternative aspect, the term ‘SASP’ as used in the specification refers to any protein listed in appendix 1 of WO02/40678.

It is preferred that the SASP gene is under the control of a constitutive promoter which is advantageously sufficiently strong to drive production of toxic levels of SASP when the modified bacteriophage is present in multiple copies in the target bacterium. Useful constitutive promoters include pdhA for pyruvate dehydrogenase E1 component alpha sub units, rpsB for the 30S ribosomal protein S2, pgi for glucose-6-phosphate isomerase and the fructose bisphosphate aldolase gene promoter fda. Preferred regulated promoters, active during infection, are lasB for elastase. These promoters are typically from P. aeruginosa. Promoters having a sequence showing at least 90% sequence identity to these promoter sequences may also be used.

In a further aspect, there is provided a composition for inhibiting or preventing bacterial cell growth, which comprises a modified bacteriophage as defined herein and a carrier therefor. Such a composition may have a wide range of uses and is therefore to be formulated according to the intended use. The composition may be formulated as a medicament, especially for human treatment and may treat various conditions, including bacterial infections. Among those infections treatable according to the present invention are localised tissue and organ infections, or multi-organ infections, including blood-stream infections, topical infections, dental carries, respiratory infections and eye infections. The carrier may be a pharmaceutically-acceptable recipient or diluent. The exact nature and quantities of the components of such compositions may be determined empirically and will depend in part upon the routes of administration of the composition.

Routes of administration to recipients include intravenous, intra-arterial, oral, buccal, sublingual, intranasal, by inhalation, topical (including ophthalmic), intra-muscular, subcutaneous and intra-articular. For convenience of use, dosages according to the invention will depend on the site and type of infection to be treated or prevented. Respiratory infections may be treated by inhalation administration and eye infections may be treated using eye drops. Oral hygiene products containing the modified bacteriophage are also provided; a mouthwash or toothpaste may be used which contains modified bacteriophage according to the invention formulated to eliminate bacteria associated with dental plaque formation.

A modified bacteriophage produced according to the invention may be used as a bacterial decontaminant, for example in the treatment of surface bacterial contamination as well as land remediation or water treatment. The bacteriophage may be used in the treatment of medical personnel and/or patients as a decontaminating agent, for example in a handwash. Treatment of work surfaces and equipment is also provided, especially that used in hospital procedures or in food preparation. One particular embodiment comprises a composition formulated for topical use for preventing, eliminating or reducing carriage of bacteria and contamination from one individual to another. This is important to limit the transmission of microbial infections, particularly in a hospital environment where bacteria resistant to conventional antibiotics are prevalent. For such a use the modified bacteriophage may be contained in Tris buffered saline or phosphate buffered saline or may be formulated within a gel or cream. For multiple use a preservative may be added. Alternatively the product may be lyophilised and excipients, for example a sugar such as sucrose may be added.

### DETAILED DESCRIPTION OF THE INVENTION

This invention will now be described in more detail, by way of example only, and with reference to the accompanying drawings, in which:

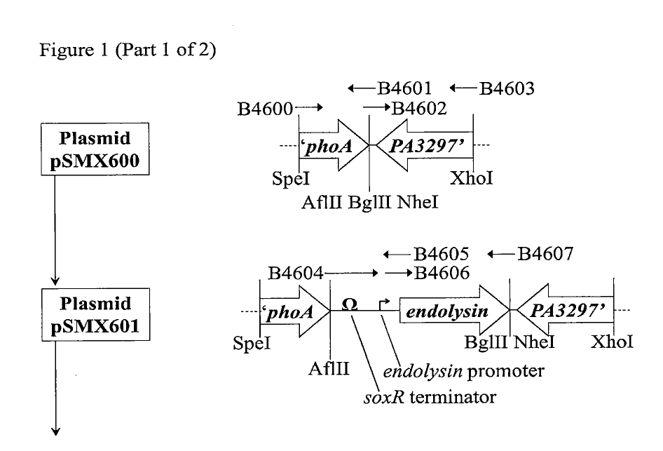

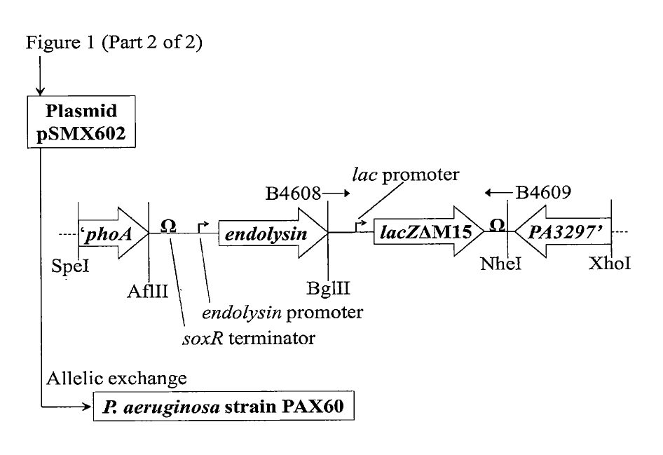

FIG. 1 is a schematic diagram showing construction of plasmids containing lacZΔM15 and Phi33 endolysin for genetic modification of P. aeruginosa to carry these genes in trans;

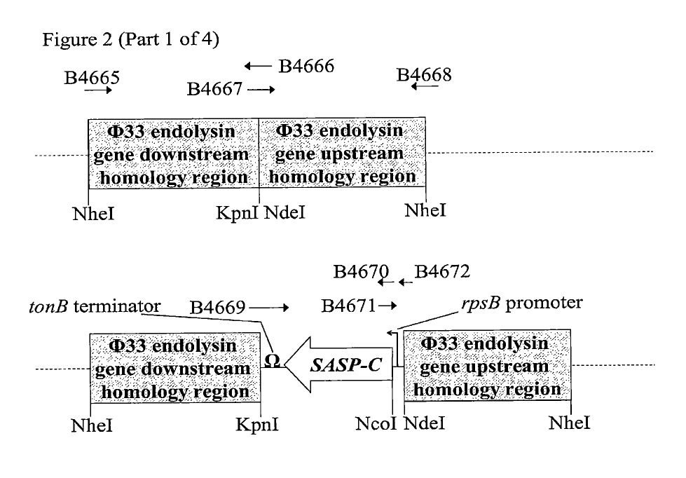

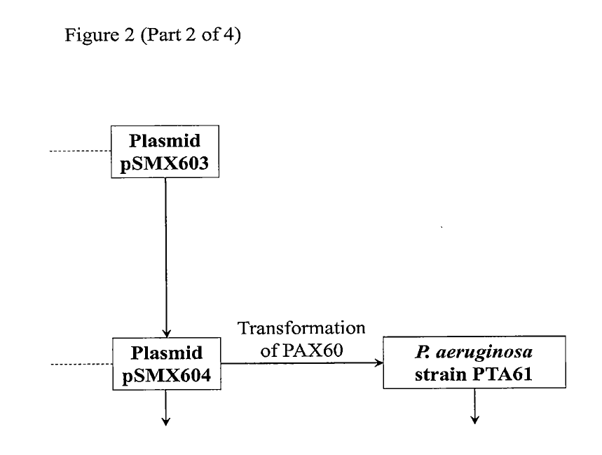

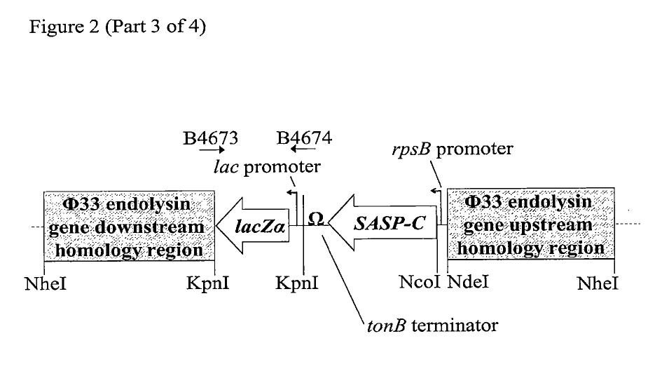

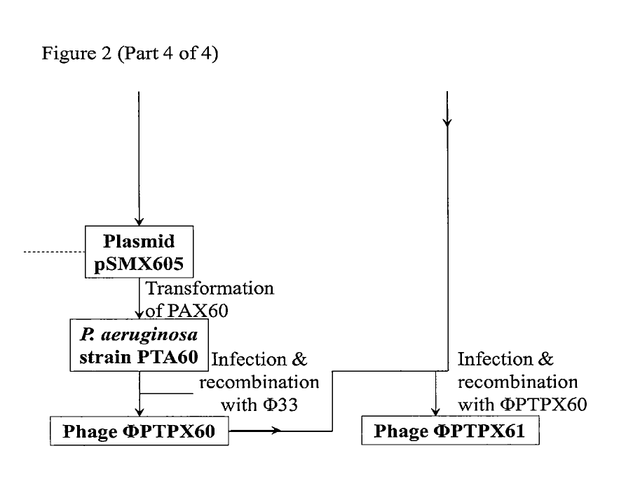

FIG. 2 is a schematic diagram showing construction of plasmids to genetically modify Phi33 to replace the endolysin gene with rpsB-SASP-C and lacZα, and then to subsequently remove the lacZα marker;

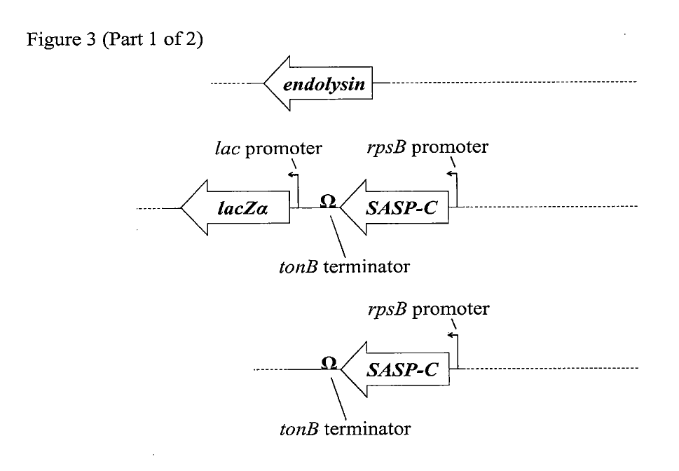

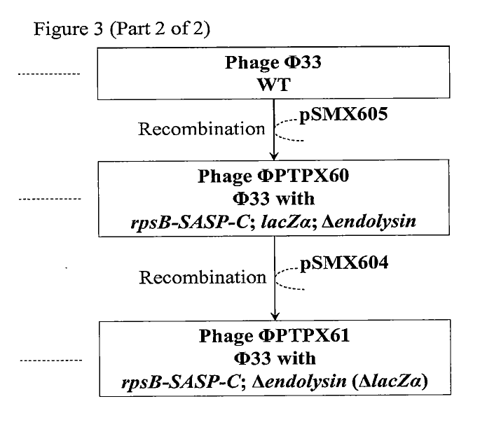

FIG. 3 is a schematic diagram showing construction of a Phi33 phage derivative which is a markerless, non-lytic phage that has endolysin replaced by rpsB-SASP-C, constructed via gain and then loss of a lacZα genetic marker, according to the invention; and

FIG. 4 shows Plate A: Recombinant 4)33 with lacZα sequence incorporated into the genome and Plate B: Wild type 4)33. In both plates, the phage were plagued on P. aeruginosa strain PAO1 expressing lacZΔM15, in the presence of S-Gal and ammonium iron III citrate.

Summary of the genetic modification of a lytic bacteriophage to render it non-lytic, and such that it carries SASP-C under the control of a promoter that usually controls expression of the 30S ribosomal subunit protein S2 gene (rpsB), utilising a lacZα marker as a means of identifying genetically-modified phage.

Genes can be removed and added to the phage genome using homologous recombination. There are several ways in which phages carrying foreign genes and promoters can be constructed and the following is an example of such methods.

For the construction of a Phi33 derivative it is shown how, using an E. coli/P. aeruginosa broad host range vector, as an example only, how the phage may be rendered non-lytic, and how the SASP-C gene under the control of an rpsB promoter may be added to the bacteriophage genome via homologous recombination, utilising a lacZα marker for the identification of recombinant phage. It is also shown how the lacZα marker may be removed via a subsequent homologous recombination step, to yield a markerless, non-lytic phage that carries the SASP-C gene under the control of an rpsB promoter.

Since these bacteriophage to be modified are lytic (rather than temperate), a requirement for these described steps of bacteriophage construction is the construction of a suitable host P. aeruginosa strain that carries both the Phi33 endolysin gene and the E. coli lacZΔM15 at a suitable location in the bacterial genome, to complement the Δendolysin, lacZΔ phenotypes of the desired recombinant bacteriophage. As an example, the construction of these P. aeruginosa strains may be achieved via homologous recombination using an E. coli vector that is unable to replicate in P. aeruginosa. The genomic location for insertion of the endolysin and lacZΔM15 transgenes should be chosen such that no essential genes are affected and no unwanted phenotypes are generated through polar effects on the expression of adjacent genes. As an example, one such location may be immediately downstream of the P. aeruginosa strain PAO1 phoA homologue.

The Phi33 endolysin gene and the E. coli lacZΔM15 allele may be cloned into an E. coli vector that is unable to replicate in P. aeruginosa, between two regions of P. aeruginosa strain PA01 genomic DNA that flank the 3′ end of phoA. This plasmid may be introduced into P. aeruginosa and isolates having undergone a single homologous recombination to integrate the whole plasmid into the genome selected according to the acquisition of tetracycline (50 μg/m1) resistance. Isolates (endolysin⁺, lacZΔM15⁺) which have undergone a second homologous recombination event may then be isolated on medium containing 10% sucrose (utilising the sacB counter-selectable marker present on the plasmid backbone).

Homologous recombination may be used to replace the endolysin gene of Phi33, to simultaneously render it non-lytic, while introducing both the gene for SASP-C, under the control of a P. aeruginosa rpsB promoter, and the E. coli lacZα genetic marker, under the control of the E. coli lac promoter. A region consisting of SASP-C controlled by the rpsB promoter, and the E. coli lacZα genetic marker controlled by the lac promoter, may be cloned between two regions of Phi33 that flank the endolysin gene, in a broad host range E. coli/P. aeruginosa vector. This plasmid may be transferred to a suitable P. aeruginosa (endolysin⁺lacZΔM15⁺) strain, and the resulting strain infected by Phi33. Progeny phage may be harvested and double recombinants identified by plaquing on P. aeruginosa (endolysin⁺lacZΔM15⁺), looking for acquisition of the lacZα reporter on medium containing a chromogenic substrate that detects the action of f3-galactosidase.

In a subsequent step, a similar homologous recombination may be used to remove the lacZα marker from the previously described, (lacZα⁺) Phi33 derivative that has been modified to replace the endolysin gene with the gene for SASP-C, under the control of a P. aeruginosa rpsB promoter. A region consisting of SASP-C controlled by the rpsB promoter, may be cloned between two regions of Phi33 that flank the endolysin gene, in a broad host range E. coli/P. aeruginosa vector. This plasmid may be transferred to a suitable P. aeruginosa (endolysin⁺lacZΔM15^{k}) strain, and the resulting strain infected by the previously described (lacZα⁺) Phi33 derivative that has been modified to replace the endolysin gene with the gene for SASP-C, under the control of a P. aeruginosa rpsB promoter. Progeny phage may be harvested and double recombinants identified by plaquing on P. aeruginosa (endolysin⁺lacZΔM15⁺), looking for loss of the lacZα reporter on medium containing a chromogenic substrate that detects the action of β-galactosidase.

### Experimental Procedures

PCR reactions to generate DNA for cloning purposes may be carried out using Herculase II Fusion DNA polymerase (Agilent Technologies), depending upon the melting temperatures (Tₘ) of the primers, according to manufacturers instructions. PCR reactions for screening purposes may be carried out using Taq DNA polymerase (NEB), depending upon the Tₘ of the primers, according to manufacturers instructions. Unless otherwise stated, general molecular biology techniques, such as restriction enzyme digestion, agarose gel electrophoresis, T4 DNA ligase-dependent ligations, competent cell preparation and transformation may be based upon methods described in Sambrook et al., (1989). Enzymes may be purchased from New England Biolabs or Thermo Scientific. DNA may be purified from enzyme reactions and prepared from cells using Qiagen DNA purification kits. Plasmids may be transferred from E. coli strains to P. aeruginosa strains by conjugation, mediated by the conjugation helper strain E. coli HB101 (pRK2013). A chromogenic substrate for β-galactosidase, S-Gal, that upon digestion by β-galactosidase forms a black precipitate when chelated with ferric iron, may be purchased from Sigma (S9811).

Primers may be obtained from Sigma Life Science. Where primers include recognition sequences for restriction enzymes, additional 2-6 nucleotides may be added at the 5′ end to ensure digestion of the PCR-amplified DNA.

All clonings, unless otherwise stated, may be achieved by ligating DNAs overnight with T4 DNA ligase and then transforming them into E. coli cloning strains, such as DH5α or TOP10, with isolation on selective medium, as described elsewhere (Sambrook et al., 1989).

An E. coli/P. aeruginosa broad host range vector, such as pSM1080, may be used to transfer genes between E. coli and P. aeruginosa. pSM1080 was previously produced by combining a broad host-range origin of replication, from a Pseudomonas plasmid, oriT from pRK2, the tetAR selectable marker for use in both E. coli and P. aeruginosa, from plasmid pRK415, and the high-copy-number, E. coli origin of replication, oriV, from plasmid pUC19.

An E. coli vector that is unable to replicate in P. aeruginosa, pSM1104, may be used to generate P. aeruginosa mutants by allelic exchange. pSM1104 was previously produced by combining oriT from pRK2, the tetAR selectable marker for use in both E. coli and P. aeruginosa, from plasmid pRK415, the high-copy-number, E. coli origin of replication, oriV, from plasmid pUC19, and the sacB gene from Bacillus subtilis strain 168, under the control of a strong promoter, for use as a counter-selectable marker.

### Construction of Plasmids to Generate a Pseudomonas Aeruginosa Strain that Carries Both the Phi33 Endolysin Gene and the Escherichia coli lacZΔM15 gene, Immediately Downstream of the phoA Locus of the Bacterial Genome

1. Plasmid pSMX600 (FIG. 1), comprising pSM1104 carrying DNA flanking the 3′ end of the P. aeruginosa PAO1 phoA homologue, may be constructed as follows.

A region comprising the terminal approximately 1 kb of the phoA gene from P. aeruginosa may be amplified by PCR using primers B4600 and B4601 (FIG. 1). The PCR product may then be cleaned and digested with SpeI and BglII. A second region comprising approximately 1 kb downstream of the phoA gene from P. aeruginosa, including the 3′ end of the PA3297 open reading frame, may be amplified by PCR using primers B4602 and B4603 (FIG. 1). This second PCR product may then be cleaned and digested with BglII and XhoI. The two digests may be cleaned again and ligated to pSM1104 that has been digested with SpeI and XhoI, in a 3-way ligation, to yield plasmid pSMX600 (FIG. 1).

Primer B4600 consists of a 5′ SpeI restriction site (underlined), followed by sequence located approximately 1 kb upstream of the stop codon of phoA from P. aeruginosa strain PAO1 (FIG. 1). Primer B4601 consists of 5′ BglII and AflII restriction sites (underlined), followed by sequence complementary to the end of the phoA gene from P. aeruginosa strain PAO1 (the stop codon is in lower case; FIG. 1). Primer B4602 consists of 5′ BglII and NheI restriction sites (underlined), followed by sequence immediately downstream of the stop codon of the phoA gene from P. aeruginosa strain PAO1 (FIG. 1). Primer B4603 consists of a 5′ XhoI restriction site (underlined), followed by sequence within the PA3297 open reading frame, approximately 1 kb downstream of the phoA gene from P. aeruginosa strain PAO1 (FIG. 1).

Primer B4600
(SEQ ID NO: 1)
5′-GATAACTAGTCCTGGTCCACCGGGGTCAAG-3′
Primer B4601
(SEQ ID NO: 2)
5′-GCTCAGATCTTCCTTAAGtcaGTCGCGCAGGTTCAG-3′
Primer B4602
(SEQ ID NO: 3)
5′-AGGAAGATCTGAGCTAGCTCGGACCAGAACGAAAAAG-3′
Primer B4603
(SEQ ID NO: 4)
5′-GATACTCGAGGCGGATGAACATTGAGGTG-3′

2. Plasmid pSMX601 (FIG. 1), comprising pSMX600 carrying the Phi33 endolysin gene under the control of an endolysin promoter, may be constructed as follows.

The endolysin promoter may be amplified by PCR from Phi33 using primers B4604 and B4605 (FIG. 1). The endolysin gene itself may be amplified by PCR from Phi33 using primers B4606 and B4607 (FIG. 1). The two PCR products may then be joined together by Splicing by Overlap Extension (SOEing) PCR, using the two outer primers, B4604 and B4607. The resulting PCR product may then be digested with AflII and BglII, and ligated to pSMX600 that has also been digested with AflII and BglII, to yield plasmid pSMX601 (FIG. 1).

Primer B4604 consists of a 5′ AflII restriction site (underlined), followed by a bi-directional transcriptional terminator (soxR terminator, 60-96 bases of Genbank accession number DQ058714), and sequence of the beginning of the Phi33 endolysin promoter region (underlined, in bold) (FIG. 1). Primer B4605 consists of a 5′ region of sequence that is complementary to the region overlapping the start codon of the endolysin gene from Phi33, followed by sequence that is complementary to the end of the endolysin promoter region (underlined, in bold; FIG. 1). Primer B4606 is the reverse complement of primer B4605 (see also FIG. 1). Primer B4607 consists of a 5′ BglII restriction site (underlined), followed by sequence complementary to the end of the Phi33 endolysin gene (FIG. 1).

Primer B4604
(SEQ ID NO: 5)
5′-GATACTTAAGAAAACAAACTAAAGCGCCCTTGTGGCGCTTTAGTTTT
ATACTACTGAGAAAAATCTGGATTC-3′
Primer B4605
(SEQ ID NO: 6)
5′-GATTTTCATCAATACTCCTGGATCCCGTTAATTCGAAGAGTCG-3′
Primer B4606
(SEQ ID NO: 7)
5′-CGACTCTTCGAATTAACGGGATCCAGGAGTATTGATGAAAATC-3′
Primer B4607
(SEQ ID NO: 8)
5′-GATAAGATCTTCAGGAGCCTTGATTGATC-3′

3. Plasmid pSMX602 (FIG. 1), comprising pSMX601 carrying lacZAM15 under the control of a lac promoter, may be constructed as follows.

The lacZΔM15 gene under the control of a lac promoter may be amplified by PCR from Escherichia coli strain DH10B using primers B4608 and B4609 (FIG. 1). The resulting PCR product may then be digested with BglII and NheI, and ligated to pSMX601 that has also been digested with BglII and NheI, to yield plasmid pSMX602 (FIG. 1).

Primer B4608 consists of a 5′ BglII restriction site (underlined), followed by sequence of the lac promoter (FIG. 1). Primer B4609 consists of a 5′ NheI restriction site (underlined), followed by a bi-directional transcriptional terminator and sequence complementary to the 3′ end of lacZΔM15 (underlined, in bold; FIG. 1).

Primer B4608
(SEQ ID NO: 9)
5′-GATAAGATCTGCGCAACGCAATTAATGTG-3′
Primer B4609
(SEQ ID NO: 10)
5′-GATAGCTAGCAGTCAAAAGCCTCCGGTCGGAGGCTTTTGACTTTATT
TTTGACACCAGACCAAC-3′

### Genetic Modification of Pseudomonas Aeruginosa to Introduce the Phi33 Endolysin Gene and Escherichia coli lacZΔM15 Immediately Downstream of the phoA Locus of the Bacterial Genome

1. Plasmid pSMX602 (FIG. 1) may be transferred to P. aeruginosa by conjugation, selecting for primary recombinants by acquisition of resistance to tetracycline (50 μg/m1).

2. Double recombinants may then be selected via sacB-mediated counter-selection, by plating onto medium containing 10% sucrose.

3. Isolates growing on 10% sucrose may then be screened by PCR to confirm that the endolysin gene and lacZΔM15 have been introduced downstream of the P. aeruginosa phoA gene.

4. Following verification of an isolate (PAX60), this strain may then be used as a host for further modification of Phi33, or similar bacteriophage, where complementation of both an endolysin mutation and a lacZα reporter are required.

### Construction of a Plasmid to Replace the Endolysin Gene of Phi33 and Similar Phage, by rpsB-SASP-C and lacZα

1. Plasmid pSMX603 (FIG. 2), comprising pSM1080 containing regions of Phi33 flanking the endolysin gene, may be constructed as follows.

The region of Phi33 sequence immediately downstream of the endolysin gene may be amplified by PCR using primers B4665 and B4666 (FIG. 2). This PCR product may then be cleaned and digested with NdeI and NheI. The region of Phi33 sequence immediately upstream of the endolysin gene may be amplified by PCR using primers B4667 and B4668 (FIG. 2). This second PCR product may then be cleaned and digested with NdeI and NheI. The two PCR product digests may then be cleaned again and ligated to pSM1080 that has been digested with NheI and treated with alkaline phosphatase prior to ligation. Clones carrying one insert of each of the two PCR products may be identified by PCR using primers B4665 and B4668, and NdeI restriction digest analysis of the purified putative clones, to identify plasmid pSMX603 (FIG. 2).

Primer B4665 consists of a 5′ NheI restriction site (underlined), followed by Phi33 sequence located approximately 340bp downstream of the Phi33 endolysin gene (FIG. 2). Primer B4666 consists of 5′ NdeI and KpnI restriction sites (underlined), followed by sequence of Phi33 that is located immediately downstream of the endolysin gene (FIG. 2). Primer B4667 consists of a 5′ NdeI restriction site (underlined), followed by sequence that is complementary to sequence located immediately upstream of the Phi33 endolysin gene (FIG. 2). Primer B4668 consists of a 5′ NheI site (underlined), followed by Phi33 sequence that is located approximately 340 bp upstream of the endolysin gene (FIG. 2).

Primer B4665
(SEQ ID NO: 11)
5′-GATAGCTAGCTTGGCCAGAAAGAAGGCG-3′
Primer B4666
(SEQ ID NO: 12)
5′-GATACATATGTCGGTACCTATTCGCCCAAAAGAAAAG-3′
Primer B4667
(SEQ ID NO: 13)
5′-GATACATATGTCAATACTCCTGATTTTTG-3′
Primer B4668
(SEQ ID NO: 14)
5′-GATAGCTAGCAATGAAATGGACGCGGATC-3′

2. Plasmid pSMX604 (FIG. 2), comprising pSMX603 containing SASP-C under the control of an rpsB promoter, may be constructed as follows.

The SASP-C gene from Bacillus megaterium strain KM (ATCC 13632) may be amplified by PCR using primers B4669 and B4670 (FIG. 2). The resulting PCR product may then be digested with KpnI and NcoI. The rpsB promoter may be amplified by PCR from P. aeruginosa using primers B4671 and B4672 (FIG. 2). The resulting PCR product may then be digested with NcoI and NdeI. The two digested PCR products may then be cleaned and ligated to pSMX603 that has been digested with KpnI and NdeI, yielding plasmid pSMX604 (FIG. 2).

Primer B4669 comprises a 5′ KpnI restriction site, followed by 5 bases, and then a bi-directional transcriptional terminator, and then sequence complementary to the 3′ end of the SASP-C gene from B. megaterium strain KM (ATCC 13632) (underlined, in bold; FIG. 2). Primer B4670 comprises a 5′ NcoI restriction site (underlined), followed by sequence of the 5′ end of the SASP-C gene from B. megaterium strain KM (ATCC 13632) (FIG. 2). Primer B4671 comprises a 5′ NcoI restriction site (underlined), followed by sequence complementary to the end of the rpsB promoter from P. aeruginosa PAO1 (FIG. 2). Primer B4672 comprises a 5′ NdeI restriction site (underlined), followed by sequence of the beginning of the rpsB promoter from P. aeruginosa PAO1 (FIG. 2).

Primer B4669
(SEQ ID NO: 15)
5′-GATAGGTACCGATCTAGTCAAAAGCCTCCGACCGGAGGCTTTTGACT
TTAGTACTTGCCGCCTAG-3′
Primer B4670
(SEQ ID NO: 16)
5′-GATACCATGGCAAATTATCAAAACGCATC-3′
Primer B4671
(SEQ ID NO: 17)
5′-GATACCATGGTAGTTCCTCGATAAGTCG-3′
Primer B4672
(SEQ ID NO: 18)
5′-GATACATATGCCTAGGGATCTGACCGACCGATCTACTCC-3′

3. pSMX605 (FIG. 2), comprising pSMX604 containing lacZα, may be constructed as follows.

lacZα may be PCR amplified using primers B4673 and B4674 (FIG. 2). The resulting PCR product may then be digested with KpnI and ligated to pSMX604 that has also been digested with KpnI and treated with alkaline phosphatase prior to ligation, to yield pSMX605 (FIG. 2).

Primer B4673 consists of a 5′ KpnI restriction site (underlined), followed by sequence complementary to the 3′ end of lacZα (FIG. 2). Primer B4674 consists of a 5′ KpnI restriction site (underlined), followed by sequence of the lac promoter driving expression of lacZα (FIG. 2).

Primer B4673
(SEQ ID NO: 19)
5′-GATAGGTACCTTAGCGCCATTCGCCATTC-3′
Primer B4674
(SEQ ID NO: 20)
5′-GATAGGTACCGCGCAACGCAATTAATGTG-3′

### Genetic Modification of Phi33 and Similar Phage, to Replace the Endolysin Gene with rpsB-SASP-C and lacZα

1. Plasmid pSMX605 (FIG. 2) may be introduced into P. aeruginosa strain PAX60 by conjugation, selecting transconjugants on the basis of tetracycline resistance (50 μg/m1), yielding strain PTA60.

2. Strain PTA60 may be infected in individual experiments with phage Phi33, or similar phage, and the progeny phage harvested.

3. Recombinant phage, in which the endolysin gene has been replaced by rpsB-SASP-C and lacZα, may be identified by plaquing the lysate from step (2) on P. aeruginosa strain PAX60, onto medium containing S-Gal, looking for black plaques, which are indicative of β-galactosidase activity.

4. PCR may be carried out to check that the endolysin gene has been replaced, and that rpsB-SASP-C and lacZα are present.

5. Following identification of a verified isolate (PTPX60; FIG. 3), the isolates may be plaque purified twice more on P. aeruginosa strain PAX60, prior to further use.

### Genetic Modification to Remove the lacZα Marker from PTPX60, to Generate a Markerless Version of Phi33, which has been Rendered Non-Lytic, and which Carries SASP-C Under the Control of an rpsB Promoter

1. Plasmid pSMX604 (FIG. 2) may be introduced into P. aeruginosa strain PAX60 by conjugation, selecting transconjugants on the basis of tetracycline resistance (50 μg/ml), yielding strain PTA61.

2. Strain PTA61 may be infected in individual experiments with phage PTPX60 (FIG. 3) and the progeny phage harvested.

3. Recombinant phage, in which lacZα marker has been removed, may be identified by plaquing the lysate from step (2) on P. aeruginosa strain PAX60, onto medium containing S-Gal, looking for clear plaques, which are indicative of loss of β-galactosidase activity.

4. PCR may be carried out to confirm removal of the lacZα marker, while ensuring that rpsB-SASP-C is still present.

5. Following identification of a verified isolate (PTPX61; FIG. 3), the isolate may be plaque purified twice more on P. aeruginosa strain PAX60, prior to further use.

### REFERENCES

Abedon S T. (2008). Bacteriophage Ecology: Population Growth, Evolution, an Impact of Bacterial Viruses. Cambridge. Cambridge University Press. Chapter 1.
Brabban A D, Hite E, Callaway T R. (2005). Evolution of Foodborne Pathogens via Temperate Bacteriophage-Mediated Gene Transfer. Foodborne Pathogens and Disease. 2:287-303.
Dobozi-King M, Seo S, Kim J U, Young R, Cheng M, Kish L B. (2005). Rapid detection and identification of bacteria: SEnsing of Phage-Triggered Ion Cascade (SEPTIC). Journal of Biological Physics and Chemistry. 5:3-7.
Francesconi, S. C MacAlister, T. J., Setlow, B., & Setlow, P. (1988). Immunoelectron microscopic localization of small, acid-soluble spore proteins in sporulating cells of Bacillus subtilis. J. Bacteriol., 170: 5963-5967.
Frenkiel-Krispin, D., Sack, R., Englander, J., Shimoni, E., Eisenstein, M., Bullitt, E. & Wolf, S. G. (2004). Structure of the DNA-SspC complex: implications for DNA packaging, protection, and repair in bacterial spores. J. Bacteriol. 186:3525-3530.
Harper DR, Anderson J, Enwright MC. (2011). Phage therapy: delivering on the promise. Ther Deliv. 2:935-47.
Hendrix RW. (2009). Jumbo Bacteriophages. Curr. Topics Microbiol. Immunol. 328:229-40.
Lee, K. S., Bumbaca, D., Kosman, J., Setlow, P., & Jedrzejas, M. J. (2008). Structure of a proteinDNA complex essential for DNA protection in spores of Bacillus species. Proc. Natl. Acad. Sci. 105:2806-2811.
Mark DF, Richardson CC. (1976). Escherichia coli thioredoxin: a subunit of bacteriophage T7 DNA polymerase. Proc. Natl. Acad. Sci. USA. 73:780-4.
Marinelli L J, Piuri M, Swigonová Z, Balachandran A, Oldfield LM, van Kessel J C, Hatfull GF. (2008). BRED: a simple and powerful tool for constructing mutant and recombinant bacteriophage genomes. PLoS One. 3:e3957.
Nicholson WL, Setlow B, Setlow P. (1990). Binding of DNA in vitro by a small, acid-soluble spore protein from Bacillus subtilis and the effect of this binding on DNA topology. J. Bacteriol. 172:6900-6906.
Pouillot F, Blois H, and Iris F. (2010). Biosecurity and Bioterrorism: Biodefense Strategy, Practice, and Science. Biosecurity and Bioterrorism. 8: 155-169
Qimron U, Marintcheva B, Tabor S, Richardson CC. (2006). Genomewide screens for Escherichia coli genes affecting growth of T7 bacteriophage. Proc. Natl. Acad. Sci. U S A. 103:19039-44.
Sheng Y, Mancino V, Birren B. (1995). Transformation of Escherichia coli with large DNA molecules by electroporation. Nucl. Acids Res. 23:1990-6.
Smith GP, Petrenko VA. (2005). Phage Display. Chem. Rev. 97:391-410.
Thomason LC Sawitzke JA, Li X, Costantino N, Court DL. (2014). Recombineering: genetic engineering in bacteria using homologous recombination. Curr. Protoc. Mol. Biol. 106:1-16.
Waldor M, Mekalanos J. (1996). Lysogenic conversion by a filamentous phage encoding cholera toxin. Science. 272:1910-1914.
Williamson S J, Houchin L A, McDaniel L, Paul J H. (2002). Seasonal variation in lysogeny as depicted by prophage induction in Tampa Bay, Florida. Appl. Environ. Microbiol. 68:4307-14.
Yu D, Ellis H M, Lee EC, Jenkins N A, Copeland N G, et al. (2000). An efficient recombination system for chromosome engineering in Escherichia coli. Proc. Natl. Acad. Sci. USA. 97:5978-5983.
Welply J K, Fowler A V, Zabin I. beta-Galactosidase alpha-complementation (1981). Overlapping sequences. J. Biol. Chem. 256:6804-10

1-19. (canceled)

20. A method for modifying the genome of a target phage, which comprises
(a) providing a vector which contains a phage-targeting region which comprises a phage genome modifying element and encodes β-galactosidase or a subunit thereof;
(b) mixing the vector with the target phage so as to modify the genome of the target phage;
(c) propagating the resultant phage on a reporter host cell in the presence of a β-galactosidase substrate labelled with a reporter label under conditions to release the label in the presence of β-galactosidase activity; and
(d) harvesting phage exhibiting β-galactosidase activity in the reporter host cell.

21. A method according to claim 20, wherein the target phage is a lytic phage.

22. A method according to claim 20, wherein the mixing of the vector with the target phage takes place in a host cell infected by the target phage.

23. A method according to claim 20, wherein the target phage genome includes a first target sequence and a second target sequence and the phage-targeting region of the vector is flanked by first and second flanking sequences homologous to the first and second target sequences of the target phage genome to allow recombination to take place whereby the genome of the target phage is modified.

24. A method according to claim 23, wherein the first and second target sequences of the target phage genome are non-contiguous.

25. A method according to claim 24, wherein the first and second target sequences of the target phage genome flank a phage gene or part thereof for inactivation of the gene following recombination.

26. A method according to claim 25, wherein the phage gene is a lysis gene.

27. A method according to claim 23, wherein the phage-targeting region of the vector further comprises an exogenous DNA sequence for incorporation into the genome of the target phage.

28. A method according to claim 27, wherein the exogenous DNA encodes an antibacterial protein.

29. A method according to claim 28, wherein the exogenous DNA comprises a gene encoding an α/β small acid-soluble spore protein (SASP).

30. A method according to claim 29, wherein the SASP is SASP-C.

31. A method according to claim 29, wherein the gene is under the control of a constitutive promoter.

32. A method according to claim 31, wherein the constitutive promoter is selected from pdhA, rpsB, pgi, fda, lasB and promoters having more than 90% sequence identity thereto.

33. A method according to claim 23, wherein at least one of the first and second flanking sequences contains a mutation as compared with the first and second target sequences of the target phage genome.

34. A method according to claim 33, wherein the mutation is a point mutation.

35. A method according to claim 20, wherein the phage targeting region encodes one of the alpha and gamma subunits of β-galactosidase and the reporter host cell expresses the other of the alpha and gamma subunits of β-galactosidase.

36. A method according to claim 35, wherein the phage targeting region encodes the alpha subunit of β-galactosidase.

37. A method according to claim 20, wherein the reporter label is a colourimetric label.

38. A method according to claim 20, wherein the harvested phage is treated to remove sequence encoding the β-galactosidase or subunit thereof.
